(19) United States
(12) Patent Application Publication
(10) Pub. No.: US 2025/0298167 A1
(43) Pub. Date: Sep. 25, 2025

(54) AUTOMATIC APPROACH FOR CORE-TO-LOG DEPTH MATCHING IN PRE-SALT CARBONATE RESERVOIRS

(71) Applicant: Schlumberger Technology Corporation, Sugar Land, TX (US)

(72) Inventors: Adna Grazielly Paz de Vasconcelos, Rio de Janeiro (BR); Lin Liang, Cambridge, MA (US); Luciana Velasco Medani, Rio de Janeiro (BR); Rodrigo Bittencourt de Aguiar, Rio de Janeiro (BR); Giovanna Da Fraga Carneiro, Rio de Janeiro (BR)

(21) Appl. No.: 19/085,254

(22) Filed: Mar. 20, 2025

Related U.S. Application Data
(60) Provisional application No. 63/567,577, filed on Mar. 20, 2024.

(51) Int. Cl. G01V 11/00; G01N 33/24; G06F 17/18

(52) U.S. Cl. CPC G01V 11/002; G01N 33/24; G06F 17/18

(57) ABSTRACT

A method for performing core-to-log depth matching includes receiving input data. The input data includes core data and well log data. The method also includes performing an autonomous data preprocessing procedure to standardize the core data and the well log data to determine correlations between the core data and the well log data. The method also includes performing an autonomous outlier removal procedure to address differences in acquisition methods and measurement principles of the core data and the well log data. The method also includes automatically determining normalized cross-correlations between measurements derived from the core data and measurements derived from the well log data. The method also includes automatically shifting the measurements derived from the core data to a new depth position based upon a maximum of the normalized cross-correlations.

### CROSS-REFERENCE TO RELATED APPLICATIONS

This application claims priority to U.S. Provisional Patent Application No. 63/567,577, filed on Mar. 20, 2024, which is incorporated by reference.

### BACKGROUND

Core samples and well logs may serve as sources of petrophysical measurements, each with advantages and limitations. Core samples may provide an accurate and reliable source of petrophysical measurements. Conversely, well logs may present a higher level of uncertainty while offering the advantage of covering a larger portion of the formation when compared to core samples. Core measurements tend to be acquired under controlled conditions but may be subject to irregularities due to pressure release and consequently, core expansion of the surface, among other issues. Despite these issues, in some instances, core data is considered as ground truth.

Aligning core depths with log depths poses some challenges due to measurement divergences in the acquisition of both types of data. In particular, both whole core and sidewall core (SWC) sample measured depths may differ from log data depths. This may hamper the correlation of both types of data and reduce potential value of the core data. Current depth matching approaches which are often manual and may be based on gamma ray measurements may not have a high applicability for pre-salt carbonate rocks, where conventional gamma-ray markers are absent. Additionally, manual methods may be time consuming and may be prone to bias and inconsistencies. Therefore, what is needed is an improved system and method for core-to-log depth matching (e.g., in pre-salt carbonate reservoirs).

### SUMMARY

A method for performing core-to-log depth matching is disclosed. The method includes receiving input data. The input data includes core data and well log data. The core data is measured from samples acquired by a first downhole tool within a wellbore. The well log data is measured by sensors on a second downhole tool within the wellbore. The method also includes performing an autonomous data preprocessing procedure to standardize the core data and the well log data to determine correlations between the core data and the well log data. The method also includes performing an autonomous outlier removal procedure to address differences in acquisition methods and measurement principles of the core data and the well log data. The method also includes automatically determining normalized cross-correlations between measurements derived from the core data and measurements derived from the well log data based upon the correlations between the core data and the well log data. The normalized cross-correlations are determined within a predetermined depth shift interval in the wellbore. The method also includes automatically shifting the measurements derived from the core data to a new depth position based upon a maximum of the normalized cross-correlations, to complete the core-to-log depth matching with no user intervention.

It will be appreciated that this summary is intended merely to introduce some aspects of the present methods, systems, and media, which are more fully described and/or claimed below. Accordingly, this summary is not intended to be limiting.

### BRIEF DESCRIPTION OF THE DRAWINGS

The accompanying drawings, which are incorporated in and constitute a part of this specification, illustrate embodiments of the present teachings and together with the description, serve to explain the principles of the present teachings. In the figures:

FIGS. 1A-1E illustrate an overview of the autonomous core data and the well log data preprocessing procedure and the outlier removal procedure, according to an embodiment.

FIGS. 2A-2C illustrate core data points from three samples moving up or down, as per the well log data used as reference, according to an embodiment.

FIG. 3A illustrates side wall core (SWC) sampling process, and FIG. 3B illustrates a core plug sampling process, according to an embodiment.

FIG. 4A illustrates SWC sampling frequency and distribution along the well, and FIG. 4B illustrates core plug sampling frequency and distribution along the well, according to an embodiment.

FIG. 5A illustrates a histogram of core porosity in (m³/m³), and FIG. 5B illustrates a histogram of log porosity in (m³/m³), according to an embodiment.

FIG. 6 illustrates a histogram showing an applied shift (cm) for the 84 sample groups, according to an embodiment.

FIGS. 7A-7D illustrate cross-plots of SWC porosity (m³/m³) versus log porosity (m³/m³), according to an embodiment.

FIGS. 8A-8D illustrate density cross-plots of SWC porosity (m³/m³) versus log porosity (m³/m³), according to an embodiment.

FIGS. 9A-9F illustrate cross-plots of core plug porosity (m³/m³) versus log porosity (m³/m³), according to an embodiment.

FIGS. 10A-10F illustrate density cross-plots of core plug porosity (m³/m³) versus log porosity (m³/m³), according to an embodiment.

FIG. 11 illustrates a flowchart of a method for performing core-to-log depth matching, according to an embodiment.

FIGS. 12A and 12B illustrate well log data resampling to allow that, at each 1 cm shift applied to the core data, it can be correlated with measurements derived from the well log data at the same depth, according to an embodiment.

FIG. 13 illustrates two different groups of two types of samples (e.g., sidewall cores and core plugs), according to an embodiment.

### DETAILED DESCRIPTION

Reference will now be made in detail to embodiments, examples of which are illustrated in the accompanying drawings and figures. In the following detailed description, numerous specific details are set forth in order to provide a thorough understanding of the invention. However, it will be apparent to one of ordinary skill in the art that the invention may be practiced without these specific details. In other instances, well-known methods, procedures, components, circuits, and networks have not been described in detail so as not to unnecessarily obscure aspects of the embodiments.

It will also be understood that, although the terms first, second, etc. may be used herein to describe various elements, these elements should not be limited by these terms. These terms are only used to distinguish one element from another. For example, a first object or step could be termed a second object or step, and, similarly, a second object or step could be termed a first object or step, without departing from the scope of the present disclosure. The first object or step, and the second object or step, are both, objects or steps, respectively, but they are not to be considered the same object or step.

The terminology used in the description herein is for the purpose of describing particular embodiments and is not intended to be limiting. As used in this description and the appended claims, the singular forms “a,” “an” and “the” are intended to include the plural forms as well, unless the context clearly indicates otherwise. It will also be understood that the term “and/or” as used herein refers to and encompasses any possible combinations of one or more of the associated listed items. It will be further understood that the terms “includes,” “including,” “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Further, as used herein, the term “if” may be construed to mean “when” or “upon” or “in response to determining” or “in response to detecting,” depending on the context.

Attention is now directed to processing procedures, methods, techniques, and workflows that are in accordance with some embodiments. Some operations in the processing procedures, methods, techniques, and workflows disclosed herein may be combined and/or the order of some operations may be changed.

This disclosure provides a procedure called core-to-log depth matching to determine the actual depths of core samples and to adjust them to the log depths. More particularly, this disclosure provides a system and method for providing an automatic approach for core-to-log depth matching in pre-salt carbonate reservoirs. This may provide an automatic and robust depth matching process that accounts for the inherent heterogeneity of pre-salt carbonate formations. This may also provide a functionality of increasing the value of pre-salt carbonate rock samples by reducing associated uncertainties related to their depth.

The automated depth-matching method may include a computer-implemented execution of an algorithm that includes three steps that are performed on (1) input core data associated with core samples and (2) input well log data that is associated with a reference well. The first step includes a data processing procedure that standardizes the core data and the well log data allowing the correlation of distinct properties and units. The second step includes an outlier removal procedure that is used to mitigate scale inconsistencies between the core data and the well log data. The third step includes a final determination of a correlation between core data measurements and well log data measurements that may pertain to an optimum shift that maximizes the correlation between the core samples and the well logs.

The method may be configured to receive input data that includes the well log data associated with a reference well and the core data that is associated with core samples. The method may input the reference well log data and the core data to be shifted, as per the reference log, and a maximum shift allowed for the core samples to the depth-matching algorithm. In one embodiment, the method may perform a quality control step on the well log data to remove nonrepresentative values. For core data, in one configuration, the method may use porosity values from routine core analysis (RCA). The porosity RCA may be selected instead of core gamma ray measurements due to its largest range of values.

The maximum shift may be input to limit how many centimeters the cores may move up or down. This parameter may be set by a user in case of previous knowledge of depth uncertainty. In one configuration, inherent assumptions with respect to the input of the data to the algorithm that pertain to the accuracy of the core data and the utility of the well log data may be utilized by the system. For example, the method may interpret the core data measurements as being accurate and representative. Additionally, the method may interpret the well log data as being on depth and as a static reference. The method may also interpret the well log data as having been checked against other logging passes to mitigate stick-slip effects.

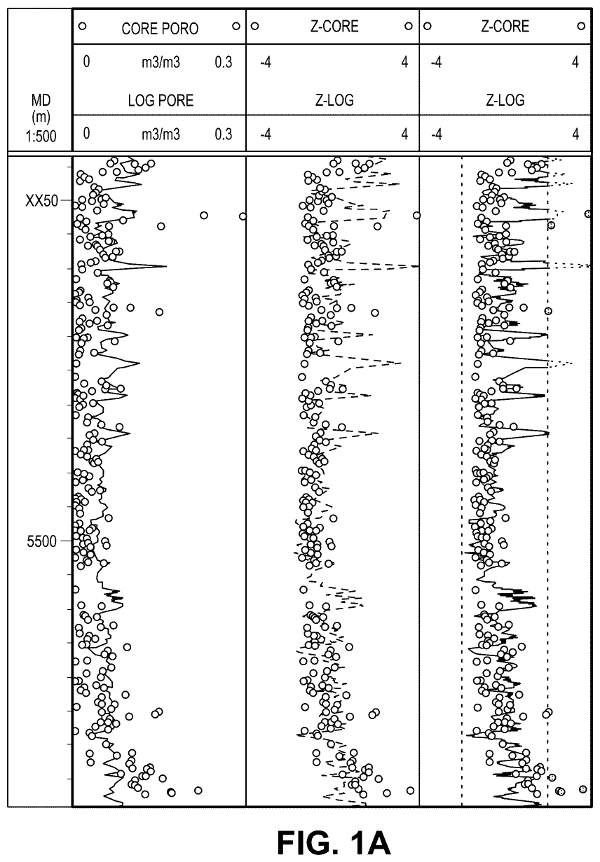

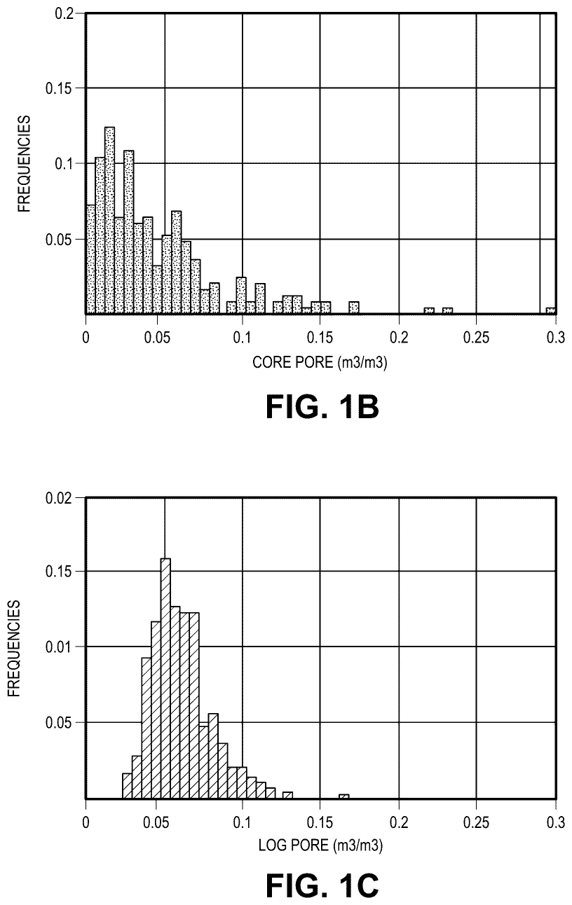

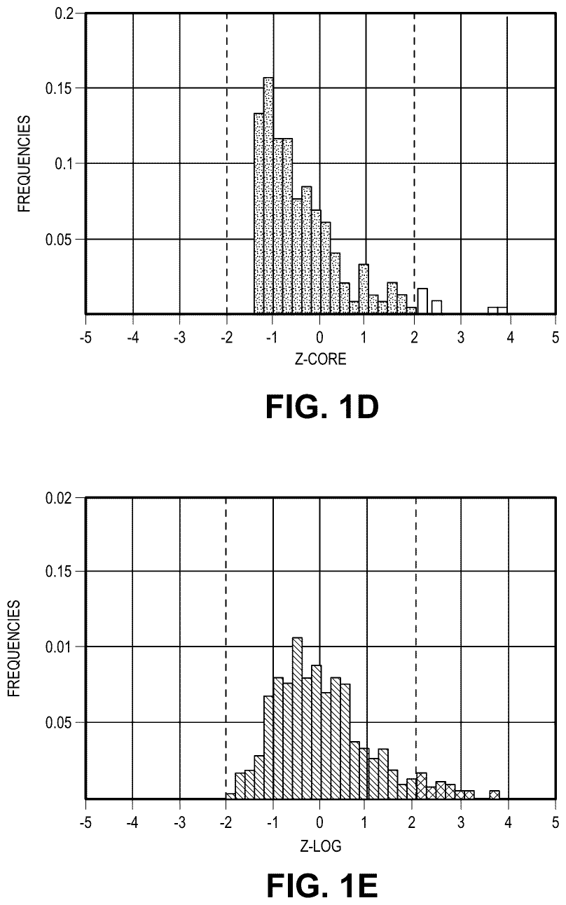

In an exemplary embodiment, with respect to the data processing procedure that standardizes the data allowing the correlation of distinct properties and units executed by the system, the method may be configured to standardize the core data associated with core samples and the well log data that is associated with a reference well. In one configuration, the method may standardize both the core data and the well log data by applying a z-score metric, described in Eq. (1):

$$Z = \frac{( x - μ )}{σ} , \qquad ( 1 )$$

where x is the value, u is the average value of the group, σ is the standard deviation, and Z is the calculated z-score value. As shown in FIGS. 1A-1E, an overview of data preprocessing and outlier removal procedures using the z-score metric is shown.

In one or more embodiments, the preprocessing step allows the depth adjustment using data from different scales and/or units to calculate the correlation between them. The data standardization methods may assume that the data have a Gaussian distribution.

With respect to the outlier removal procedure, after data standardization, the outliers may be removed from z-log and z-core data. The cutoff values for z-scores are two units of standard deviation. In one example, with the cutoff values, about 95% of the input data may still be considered if it follows a normal distribution.

With respect to determining a final computation of correlation between the core and log value, the normalized cross-correlation (NCC) step is where the shift is determined. The suggested shift of core samples may be based on the maximum correlation between values associated with the core data and the well log data. In one embodiment, the metric applied to measure the correlation between the core and log values for the NCC is defined by Eq. 2:

$$NCC = \frac{{\sum }_{d} \{ [ A ( d ) - \overline{A} ] * [ B ( d - s ) - \overline{B} ] \}}{\sqrt{{\sum }_{d} \{ {[ A ( d ) - \overline{A} ]}^{2} * {[ B ( d - s ) - \overline{B} ]}^{2} \}}} , \qquad ( 2 )$$

where A is the z-log, Ā is the average value for z-log samples, B is the z-core, B̅ is the average value for z-core samples, d is the depth, ranging from minimum to maximum registered log depth, and s is the allowed shift.

In an exemplary embodiment, the method may utilize the NCC to measure the similarities between two signals by calculating the cross correlation between them. Upon calculation of the cross correlation between the signals, the calculation may thereby be normalized to account for differences in their mean and standard deviation. In one embodiment, the method may be configured to utilize the NCC as a metric for core-to-log correlation to provide a simplified implementation and interpretation and a quantitative measure of the correlation between the core and log data, enabling an objective assessment of alignment quality.

In one configuration, core sliding may be limited by the maximum value parameterized by the user, as described above with respect to the inputting of the maximum shift. The core data may iteratively slide a predetermined number of centimeters (e.g. 1 cm), and at each shift, an NCC value may be determined, until it reaches the maximum shift parameterized by the user. The shift between the core data and the well log data may vary for each core extraction job. For example, if there are two SWC sample extraction jobs, the algorithm may be configured to find the best shift for each sample type, provided the user specifies its type, and the algorithm may be configured to find the best shift for each sample group, provided the user specifies it belongs to different core groups.

It is to be appreciated that, despite an uncertainty on the whole core length due to incomplete core recovery, the same assumption can be adapted to core plugs extracted from whole core samples. The algorithm may output the optimum shift for each sample group belonging to the same whole core. With this, core plugs derived from the same whole core may be shifted together, minimizing depth errors that may arise from core fragmentation, especially in unconsolidated formations or highly porous or fractured carbonate environments.

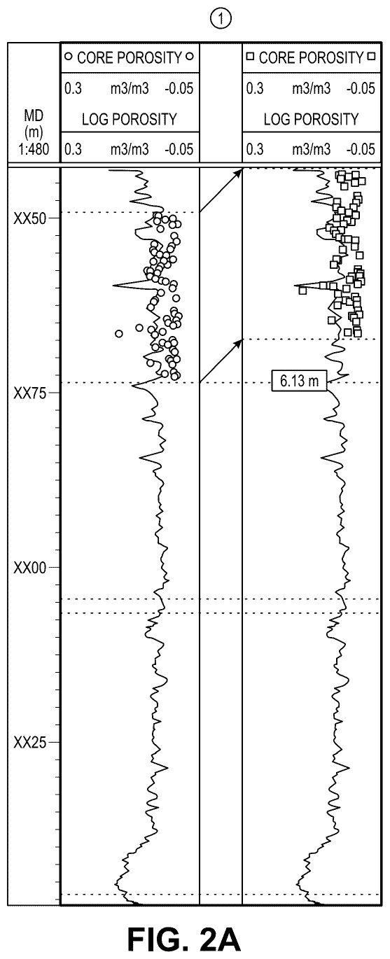

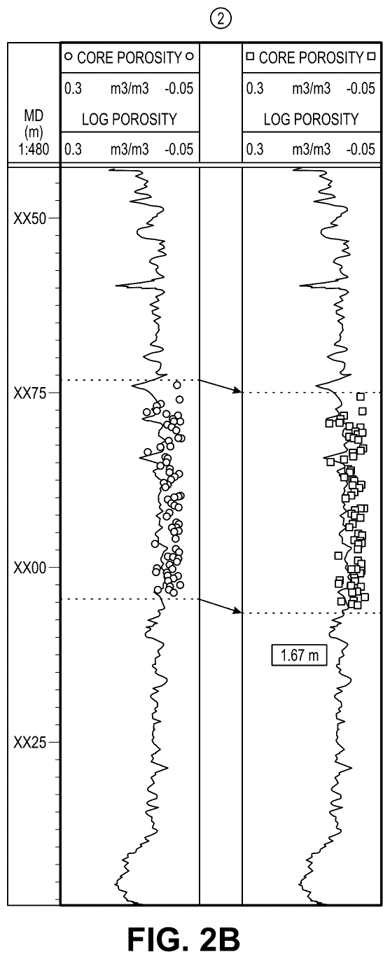

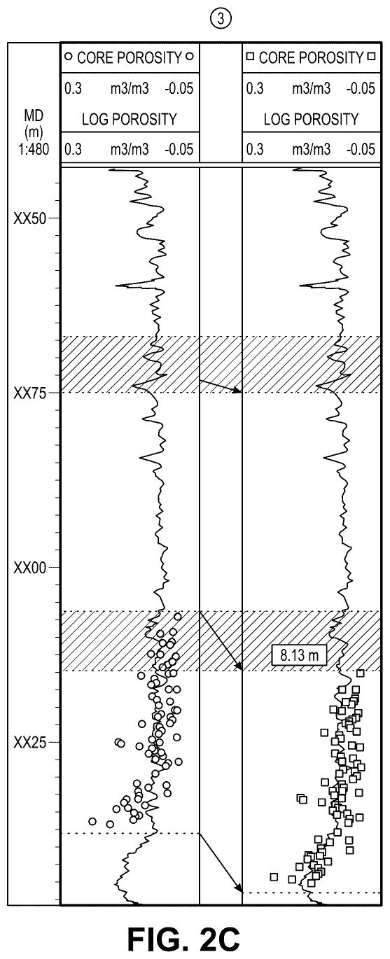

As an illustrative example, FIGS. 2A-2C illustrate the moving process for three whole cores that are extracted by the same drilling job. As shown, for the first whole core, the core points were moved up by more than 6 m. The same procedure was independently performed for the second and third whole cores, reaching a shift of 1.67 m down and more than 8 m down, respectively. The gap between the sample groups, shaded in FIGS. 2A-2C, which indicates an incomplete core recovery and loss of 7.80 m on the first interval and 8.13 m on the second interval.

Accordingly, the method provides an automatic and robust depth matching process that accounts for the inherent heterogeneity of pre-salt carbonate formations thereby minimizing the divergence with respect to the acquisition of core data and well log data. The functionality of the method may lead to lower depth positioning errors which do not involve a large depth correction to be made.

The method thereby provides an improvement in the technology with respect to core-to-log depth matching. For instance, in comparison to manual matching, the adoption of the data preprocessing step and outlier removal procedures implemented by the method may provide a more accurate and efficient measurement of the actual depth of samples with little to no user intervention. Accordingly, this improvement saves time and effort. For example, utilizing the output from the method, it may take a nominal amount of time (e.g., 1 minute) per well to automatically position the samples at their proper depth.

It is to be appreciated that the steps executed by the method may be applied to other scenarios, such as gamma ray measurements, with the advantage of being an automated approach. In addition, the core gamma ray measurements which may be acquired intended to perform depth control may be omitted for pre-salt carbonate scenarios. In some cases, this may thereby reduce the cost of laboratory measurements.

The execution of the steps discussed above by the method may thereby enhance the values of core samples for petrophysical models and may improve their overall accuracy, especially for carbonate reservoirs. Accordingly, permeability, mineralogy, and water saturation models may achieve a greater accuracy and may be extended to non-cored intervals.

### Additional Details

Core samples and well logs may serve as sources of petrophysical measurements, each with its own advantages and limitations. Despite its limitations, core data is often considered the ground truth. It may be utilized for petrophysical modeling. The core data may be the first step in linked data interpretation. However, aligning core depths with log depths still poses some challenges due to measurement divergences in the acquisition of both types of data.

Conventional depth matching approaches, often manual and based on gamma ray measurements, may not have a high applicability for pre-salt carbonate rocks, where conventional gamma-ray markers are absent. Furthermore, manual methods are time consuming and prone to bias.

By comparing petrophysical properties from a laboratory core analysis with corresponding well logs, the method described herein may develop an automatic and robust depth matching process that accounts for the inherent heterogeneity of pre-salt carbonate formations.

The proposed solution underwent validation across thousands of core samples derived from 10 challenging Brazilian pre-salt fields, highlighting an improvement in core-to-log data correlation, thus increasing the value of petrophysical data for reservoir characterization and exploration activities.

Core samples can be an accurate and reliable source of petrophysical measurements. Conversely, well logs present a higher level of uncertainty but offer the advantage of covering a larger portion of the formation when compared to cores. These two sources of information can be combined to improve formation evaluation for a more reliable formation evaluation.

Most well log measurements are indirectly obtained and are acquired under adverse conditions during the wellbore construction process, including high pressure and temperature, formation fluid substitution, and borehole wall instability. In contrast, core measurements tend to be acquired under controlled conditions but are also subject to errors due to (1) pressure release and, consequently, core expansion at the surface, (2) mistakes in core cleaning procedures, and/or (3) being nonrepresentative of the formation, by oversampling the zones with the best reservoir characteristics.

Nevertheless, despite the limitations of core-derived petrophysical properties and the high acquisition cost, a single core can be submitted to innumerable experiments to retrieve mechanical, chemical, and fluid flow properties. Most of the measurement techniques are nondestructive, and the results can be later combined with a multi-physics analysis. In addition, X-ray tomography has been widely used to create digital rock models that allow estimating several properties under countless reservoir conditions in parallel.

For this reason, the core data is assumed to be the ground truth, and it may be useful for several tasks, including to:
Calibrate log-derived properties, such as water saturation estimated from resistivity logs
Validate log interpretation methods, such as pore size distribution from nuclear magnetic resonance logs
Roughly estimate permeability and wettability
Quantify the uncertainty of log-derived properties
Find correlations between different petrophysical properties

Considering this, the core depths should be properly aligned with the well logs, to support the benchmarking step of any petrophysical modeling or interpretation task.

However, both types of data may be subject to depth measurement divergence that can reach more than 10 m, due to several reasons, including, but not limited to, inaccuracies of drill pipe length because the pipes are constantly subject to expansion-compression loads in the borehole, wireline cable stretching due to mud cakes and geometrical anomalies, and heave wave motion influence. In addition, the well logging and core extraction processes occur in distinct passes since the procedures applied and the tools used for acquisition are distinct.

As a consequence, both whole core and sidewall core (SWC) sample measured depths may differ slightly from well log data depths, hampering the correlation of both types of data and reducing the potential value of the core data. Because of that, an auxiliary procedure called core-to-log depth matching may be used to determine the actual depths of core samples and adjust them to the log depths.

Furthermore, the conventional adjustment approach may be performed manually, where processing a single exploratory well involves analyzing numerous coring intervals. This may take a petrophysicist approximately 1 hour, and may not find the best shift. Thus, the process of selecting an optimal shift may be tedious, time consuming, and prone to human bias.

There is still no option to automate core-to-log depth matches in commercially available software packages. However, this process can be automated by maximizing the correlation between log and core measurements within the possible shifts.

Thus, the method described herein may automate core-to-log depth matching by comparing petrophysical properties obtained through laboratory analysis of core data with the corresponding well logs applied to carbonate rocks. The method aims to increase the value of pre-salt carbonate rock samples by reducing the associated uncertainties related to their depth through an automatic and robust depth-matching process that considers the inherent heterogeneity of such formations.

### Methodology

The automated depth-matching method combines statistical methods and includes three steps:
A data preprocessing procedure standardizes the data allowing the correlation of distinct properties and units.
An outlier removal procedure is used to mitigate scale inconsistencies between core and log data.
A final computation of correlation between the core and log values suggests the optimum shift that maximizes the correlation between the core and log.

The details of input, the recommended practices, and the core steps of the methodology are described below.

### Input

The depth-matching algorithm inputs may include a reference well log, the core data to be shifted, as per the reference log, and the maximum shift allowed for the core samples. As previously mentioned, well logs and core samples may be extracted in different circumstances, and the analyzed properties may be obtained according to distinct physical principles and/or conditions. To deal with this great variety of scales, volumes of interest, and physical principles, some are described below.

Despite the existence of an outlier removal step, a quality control step may be performed on the well log data to remove nonrepresentative values, such as those found in washout intervals. For core data, porosity values from routine core analysis (RCA) may be used. The porosity RCA may be selected, instead of core gamma ray measurements, due to its large range of values.

The maximum shift allowed to be applied to core depths may limit how many centimeters the cores can move up or down. This parameter can be set by the user in case of previous knowledge of depth uncertainty.

The following assumptions may be made:
Core data measurements are accurate and representative
Log data are on depth and may be used as static reference
Log data are already checked against other logging passes to mitigate stick-slip effects.

### Data Standardization

The first step of the method may include a data preprocessing procedure, which standardizes both core data and well log data by applying the z-score metric, described in Eq. (1).

FIGS. 1A-1E illustrate an overview of the autonomous core data and the well log data preprocessing procedure and the outlier removal procedure, according to an embodiment. The core data is represented by a core porosity (“core pore”), and the well log data is represented by a porosity log (“log pore”). More particularly, FIG. 1A shows a log view of the porosity log and core. Track 1 shows a porosity log in the continuous line (m³/m³) and core porosity in circles (m³/m³) before data standardization. Track 2 shows z-score of porosity log in the dashed line (unitless) and z-score of core porosity in circles (unitless) after data standardization. Track 3 shows a z-score of porosity log in dashed black line (unitless), z-score of core porosity in black circles (unitless), outliers of z-score of porosity log in dashed gray line (unitless), and outliers of z-score of core porosity in gray circles (unitless). FIG. 1B shows a histogram of core porosity in (m³/m³), FIG. 1C shows a porosity log in (m³/m³). FIG. 1D shows a histogram of z-score of core porosity in (m³/m³), and FIG. 1E shows a z-score of porosity log in (unitless) with outlier values shaded in gray.

This preprocessing step allows the depth adjustment using data from different scales and/or units to calculate the correlation between them, such as bulk density (unit g/cm³) and core porosity (unit m³/m³). The data standardization methods assume the data have a Gaussian distribution.

### Outlier Removal

After data standardization, the outliers may be removed from the z-log data and the z-core data. In an example, the cutoff values for z-scores may be two units of standard deviation. FIGS. 1A-1E show the actual porosity values, the z-score for both variables, and the outliers.

### Normalized Cross Correlation

The normalized cross-correlation (NCC) step is where the shift is determined. The suggested shift of core samples may be based on the maximum correlation between the core and log values.

The metric applied to measure the correlation between core and log values is the NCC, defined by Eq. 2. The NCC measures the similarity between two signals by calculating the cross-correlation between them, which is then normalized to account for differences in their mean and standard deviation. Using the NCC as a metric for core-to-log correlation simplifies implementation and interpretation and provides a quantitative measure of the correlation between the core and log data, enabling an objective assessment of alignment quality.

The core sliding may be limited by the maximum value parametrized by the user. The core data may be iteratively slid (e.g., 1 cm), and at each shift, an NCC value may be determined, until it reaches the maximum shift allowed by the user.

The shift between core and log data can vary for each core extraction job. For example, if there are two SWC sample extraction jobs, the algorithm is designed to find the best shift for each sample group, provided the user specifies it belongs to different core groups. Despite uncertainty on whole core length due to incomplete core recovery, the same assumption can be adapted to core plugs extracted from whole core samples.

The algorithm may suggest the optimum shift for each sample group belonging to the same whole core. With this, core plugs derived from the same whole core may be shifted together, minimizing depth errors that may arise from core fragmentation, especially in unconsolidated formations or highly porous or fractured carbonate environments.

FIGS. 2A-2C illustrate core data points from three samples moving up or down, as per the well log data used as reference, according to an embodiment. The core data is represented by a core porosity (“core pore”), and the well log data is represented by a porosity log (“log porosity”). The points in the left track of each sample refer to core porosity (m³/m³) values at initial depth shift, while the solid line refers to the porosity log (m³/m³). The circles in the right track of each sample indicate the core porosity (m³/m³) values at new core depths suggested by the algorithm. This dataset has three whole core samples (1, 2, and 3), and for each sample group, a specific shift was calculated. The shaded intervals in the third sample highlight the actual core interval not recovered.

Said another way, FIGS. 2A-2C illustrate the moving process, for three whole cores, extracted by the same drilling job. For the first whole core, the core points were moved up by more than 6 m. The same procedure was independently performed for the second and third whole cores, reaching a shift of 1.67 m down and more than 8 m down, respectively. The gap between the sample groups, highlighted in shades in FIGS. 2A-2C, indicates an incomplete core recovery and loss of 7.80 m on the first interval and 8.13 m on the second interval.

### Dataset

The example dataset includes core samples and wireline openhole logs from 37 wells, from 10 Brazilian oil fields, totaling 6971 samples. Wireline openhole porosity logs were used as reference. The core measurement that was selected to be auto-adjusted was the porosity from the RCA experiment, because it better represents the pre-salt carbonates heterogeneities when compared to conventional gamma ray measurements used for this purpose.

Regarding the choice of reference logs, both neutron and nuclear magnetic resonance logs were used since they measure the same property: the amount of pore volume. Most of the porosity logs used were derived from nuclear magnetic resonance, except for three wells where neutron porosity was used as the reference.

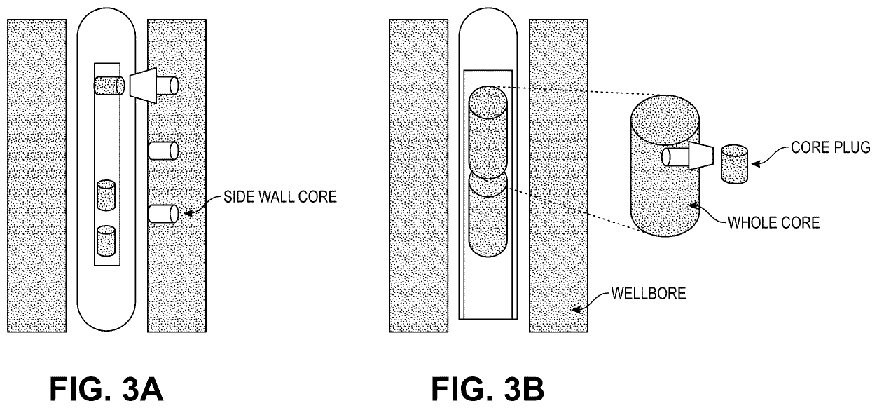

FIG. 3A illustrates side wall core (SWC) sampling process, and FIG. 3B illustrates a core plug sampling process, according to an embodiment. Two types of core data that were used as reference for depth match purposes were sidewall cores and core plugs. The SWC samples are cylindrical specimens directly extracted from borehole wall after borehole drilling (FIG. 3A), measuring approximately 1 in. in diameter and 2 in. in length.

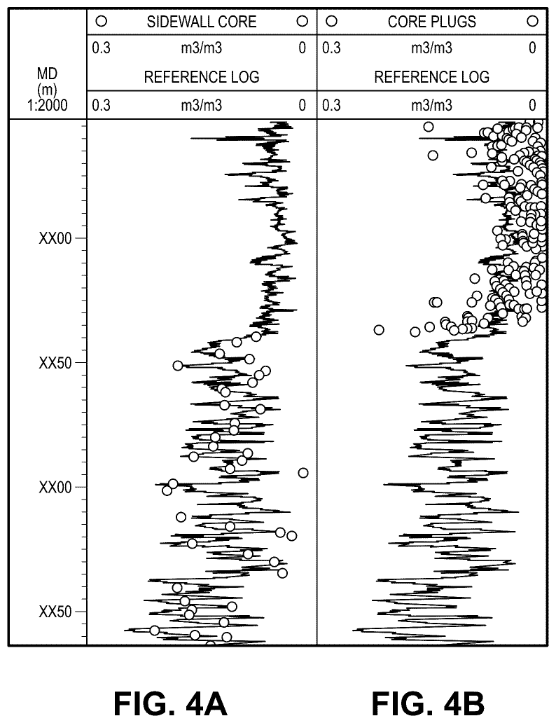

FIG. 4A illustrates SWC sampling frequency and distribution along the well, and FIG. 4B illustrates core plug sampling frequency and distribution along the well, according to an embodiment. The solid line represents the porosity log (m³/m³) used for reference depth, while the dots represent core porosity (m³/m³) from RCA that should be submitted to depth control.

The number of SWC samples depends on the coring tool capacity, and the high average time for sample extraction is another factor that limits the number of samples extracted per job. With that, the frequency of SWC sampling may be relatively low, as illustrated in FIG. 4A. Among the 37 wells, 31 were submitted to SWC sampling jobs, with one of them submitted to two coring jobs, totalizing 32 sample groups comprising 2135 samples.

Core plugs, on the other hand, are extracted from other volumes of rock, the whole core samples (FIG. 3B), and measure 1.5 to 2.5 in. in diameter and 1 to 1.5 in. in length. With that, the frequency of sidewall core sampling may be relatively low, as illustrated in FIG. 4B. The whole core samples can vary in diameter, as a function of borehole diameter and core cutting bit, and can vary in length, depending on instabilities in the coring process. In this case, the number of core plugs depends on the whole core diameter and length, and the frequency tends to be relatively higher than SWC sampling (FIG. 4B), because the whole cores are limited to a shorter and continuous interval when compared to SWCs.

Regarding core plug samples, only 19 of the 37 wells were submitted to whole core sampling jobs and, therefore, to core plug extraction. Among the 19 wells, 52 sample groups were considered, including 4836 samples.

Although both core and log data represent the same physical property (e.g., porosity, for this case study), they differ in measurement principles, data acquisition conditions, the analyzed volume, and the data resolution. Most of log data used present a sampling rate varying from 15 to 20 cm, depending on the tool, which means that a porosity value on the log curve is an average value of that interval. On the other hand, the extracted samples do not reach 10 cm length.

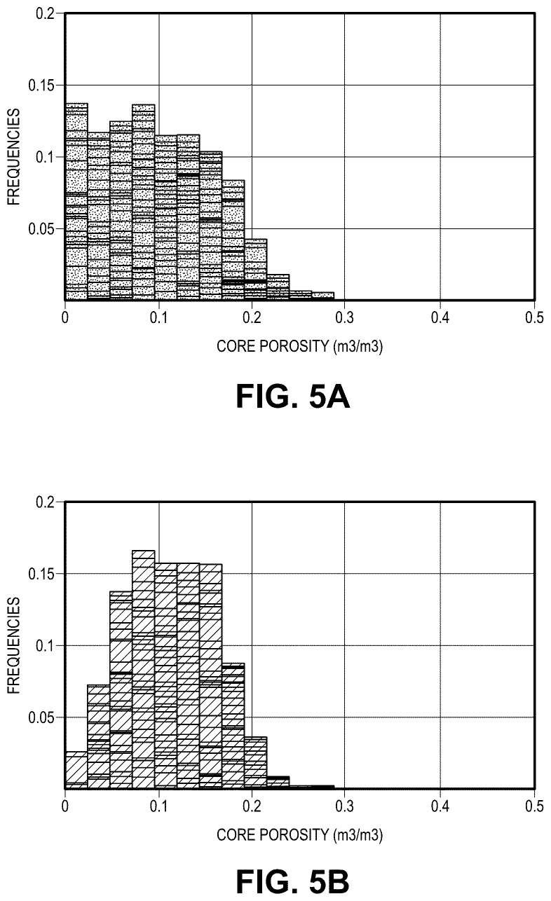

FIG. 5A illustrates a histogram of core porosity in (m³/m³), and FIG. 5B illustrates a histogram of log porosity in (m³/m³), according to an embodiment. Due to this contrast in the volume of interest, the RCA experiment tends to have lower reads of core porosity (less than 5%), as can be observed in FIGS. 5A and 5B. The core samples may capture thin low-porosity layers. In contrast, because log porosity values are an average representation of a formation interval, they are not highly affected by thin low-porosity layers.

Another point to be mentioned is the divergence of analyzed rock volumes, especially when it takes core plugs into account. Referring back to FIG. 3B, the analyzed core sample taken from the borehole (e.g., whole core) is completely separate from the volume of rock submitted to logging acquisition (e.g., borehole wall).

### Results

For validation, this automated method was applied to 37 wells located in 10 challenging Brazilian pre-salt fields. The samples are in the pre-salt carbonate interval. To measure the improvement of core-to-log correlation, metric was used to calculate the correlation: the R-squared (R2) metric. The values were calculated before and after the core depth shift, for each sample group. A correlation improvement metric was also calculated for each sample group and is given by Eq. 3, as follows:

$$correlation improvement = \frac{{R}_{after}^{2}}{{R}_{before}^{2}} - 1 , \qquad ( 3 )$$

where the R² metric, also known as the coefficient of determination, is a statistical measure that indicates how well the independent (core porosity) variables explain the variability of the dependent variable (log porosity). The R² before metric is calculated using log and core data before depth shift, while the R² after is calculated using log and core data after the automatic depth shifting process.

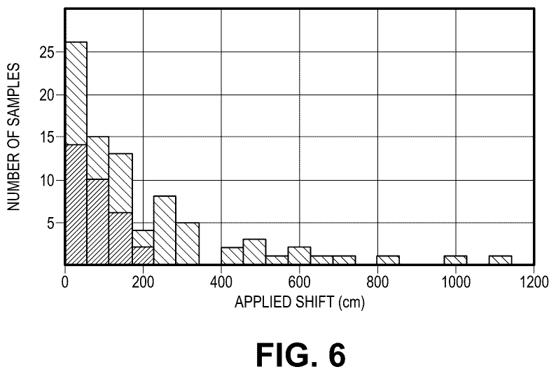

FIG. 6 illustrates a histogram showing an applied shift (cm) for the 84 sample groups, according to an embodiment. SWC sample shifts (cm) are in darker shading (i.e., in the bottom portions of the four leftmost columns), while core plug sample shifts (cm) are presented in lighter shading. In other words, the darker shading represents the SWC depth corrections, while the lighter shading represents the depth corrections applied to core plugs. The case study results were grouped by the sample-core plugs and SWC samples-given the statistical representativeness of each type of core sample.

One can notice that the depth positioning errors for SWC samples tend to be lower than depth errors for core plug samples, what may indicate a higher precision on the wireline SWC sampling tool when compared to the sum of drill pipe lengths for the whole core extraction procedure.

The results were also consolidated per shift range, adopting two categories for SWC samples: up to 1-m shift applied and between 1- and 2-m shift applied. The consolidated results of SWC shifting procedure are presented in Table 1. Although an outlier detection technique has been applied, the R² presented from now on considers each of the samples, including the outliers.

TABLE 1

| | | Number | | | | Correla- |
|---|---|---|---|---|---|---|
| Sample | Sample | of SWC | Shift | R² | R² | tion im- |
| type | groups | samples | category | before | after | provement |
| SWC | 22 | 1434 | Up to 1 m | 0.3106 | 0.4121 | 32.67% |
| samples | 10 | 701 | between | 0.2584 | 0.3288 | 27.24% |
| | | | 1 m and | | | |
| | | | 2 m | | | |

The results show that most of the SWC samples can use a depth shift up to 1 m. The SWC samples shifted by up to 1 m show an average correlation improvement of about 33%, and for those SWC samples with a depth shift between 1 and 2 m, the correlation improvement was even higher, reaching 27%, when compared to the core-to-log correlation of samples without depth correction.

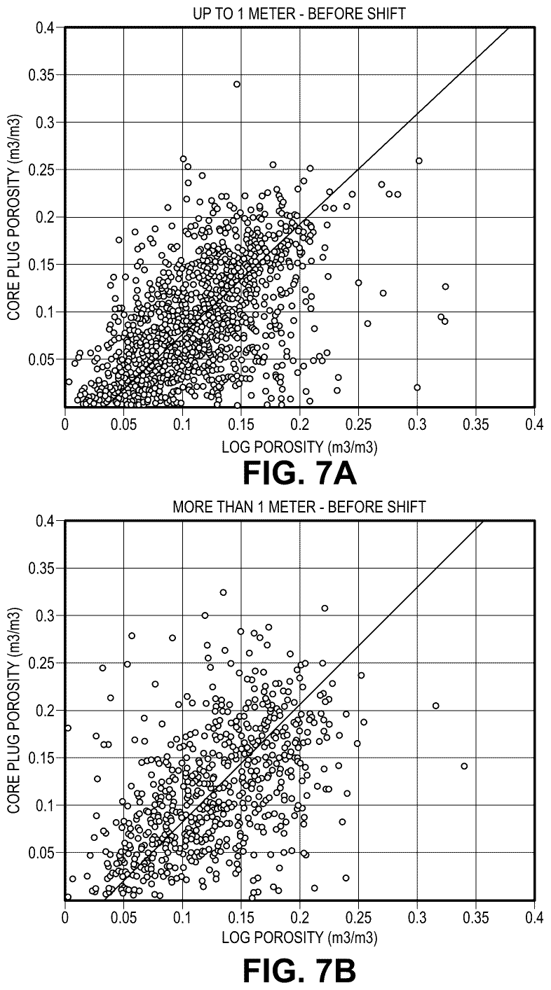

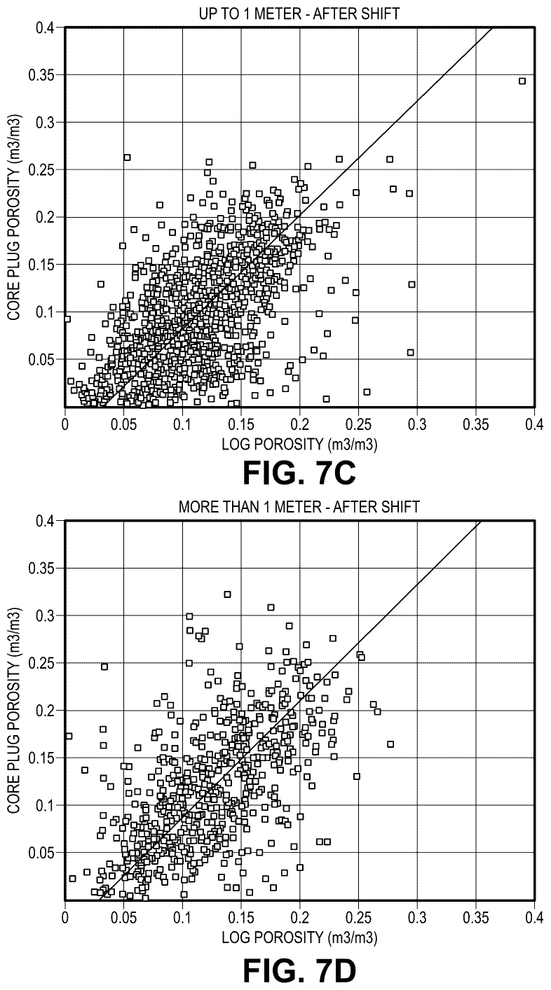

FIGS. 7A-7D illustrate cross-plots of SWC porosity (m³/m³) versus log porosity (m³/m³), according to an embodiment. More particularly, FIG. 7A shows samples shifted by up to 1 m before, FIG. 7C shows samples shifted by up to 1 m after automated core-to-log depth shift. FIG. 7B shows samples shifted by more than 1 m before, and FIG. 7D shows samples shifted by more than 1 m after automated core-to-log depth shift.

Due to the differences in scales discussed previously, for porosity values less than 20%, the SWC core measurements tend to be lower than the log measurement at the same depth. The slope of data regression reveals the tendency to not sample low porosity thin layers or zones with pores bigger than core size. When the cross-plots before (FIGS. 7A and 7B) and after (FIGS. 7C and 7D) the automated core-to-log depth matching are compared, it may be seen that there is less spread in the points than at the original depth.

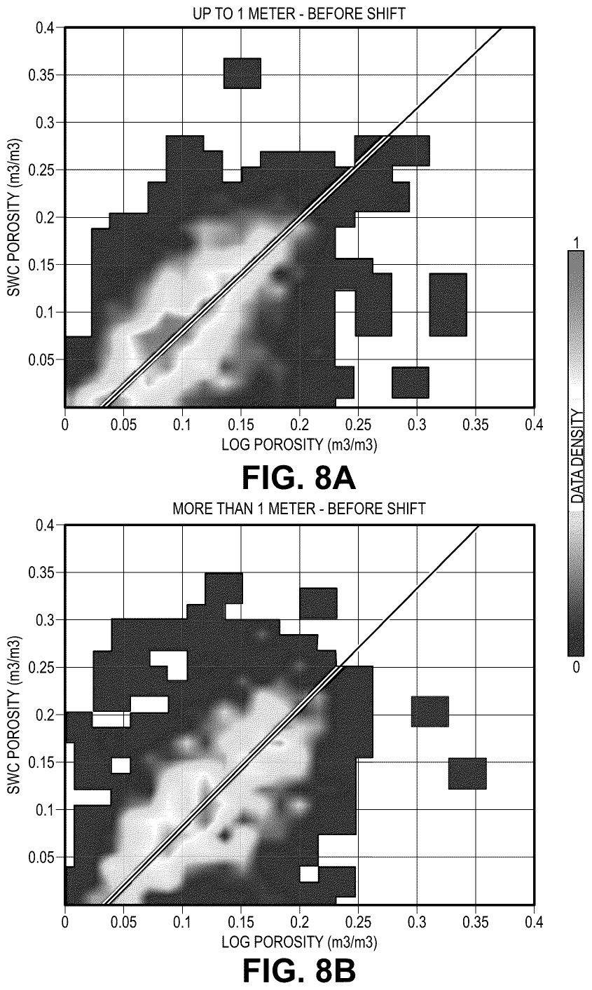

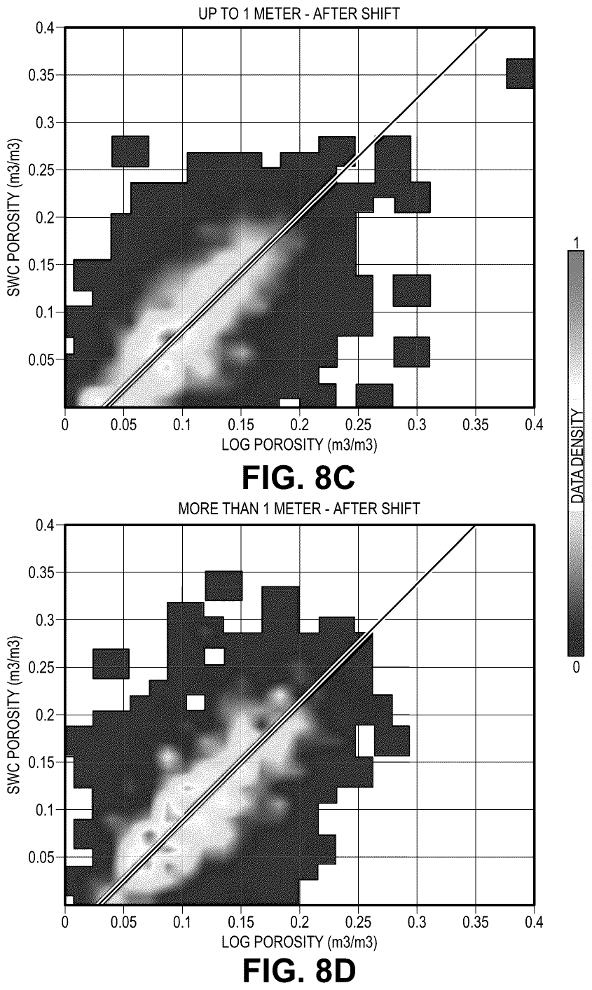

FIGS. 8A-8D illustrate density cross-plots of SWC porosity (m³/m³) versus log porosity (m³/m³), according to an embodiment. More particularly, FIG. 8A shows samples shifted by up to 1 m before automated core-to-log depth shift, and FIG. 8C shows samples shifted by up to 1 m after automated core-to-log depth shift. FIG. 8B shows samples shifted by more than 1 m before automated core-to-log depth shift, and FIG. 8D shows samples shifted by more than 1 m after automated core-to-log depth shift. There is a high spread of data on cross-plots of SWC porosity versus log porosity before depth correction (FIGS. 8A and 8B).

For core plug samples, an extra shift range was adopted, with more than 2 m shift applied. The consolidated statistics for correlation improvement achieved by the core plug shifting procedure are presented in Table 2.

TABLE 2

| | | Number of | | | | Correla- |
|---|---|---|---|---|---|---|
| Sample | Sample | core plug | Shift | R² | R² | tion im- |
| type | groups | samples | category | before | after | provement |
| Core | 14 | 1083 | Up to 1 m | 0.3771 | 0.4362 | 15.67% |
| plug | 10 | 991 | Between 1 | 0.3235 | 0.5875 | 81.62% |
| samples | | | m and 2 m | | | |
| | 28 | 2762 | More than | 0.0966 | 0.3351 | 246.93% |
| | | | 2 m | | | |

The results show that most of core plug samples can use a large depth shift (e.g., greater than 2 m), indicating a low precision on depth measurements for whole core extraction tools, when compared to SWC sampling tools. Almost 70% of SWC samples can use less than 1 m depth shift while more than 50% of core plug samples can use more than 2 m depth correction.

The core plug samples shifted up to 1 m show an average correlation improvement of 16%, for those with depth shift between 1 and 2 m, the correlation improvement was even higher, reaching about 82%. Finally, for those samples moved by more than 2 m, the correlation improvement reached a 247% increase, when compared to the core-to-log correlation of samples without depth correction.

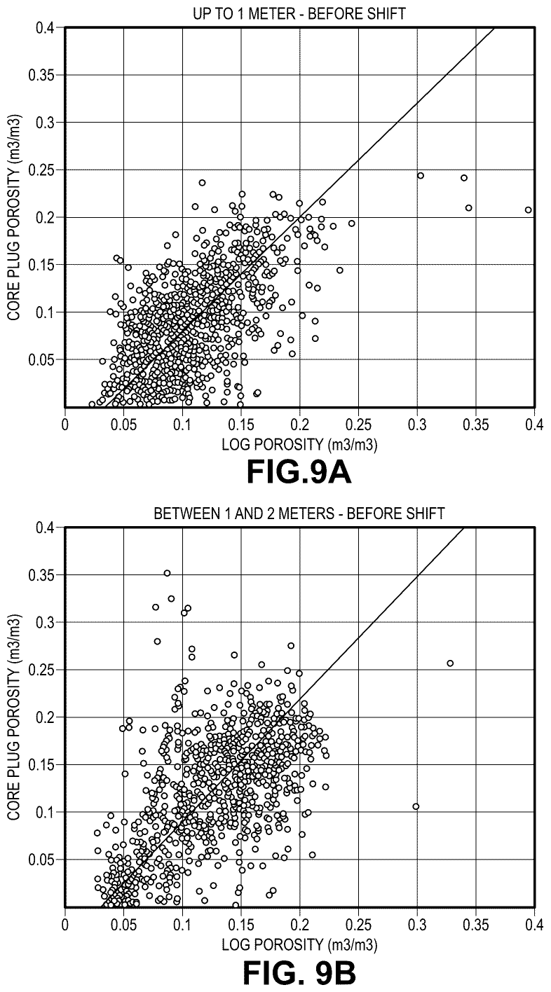

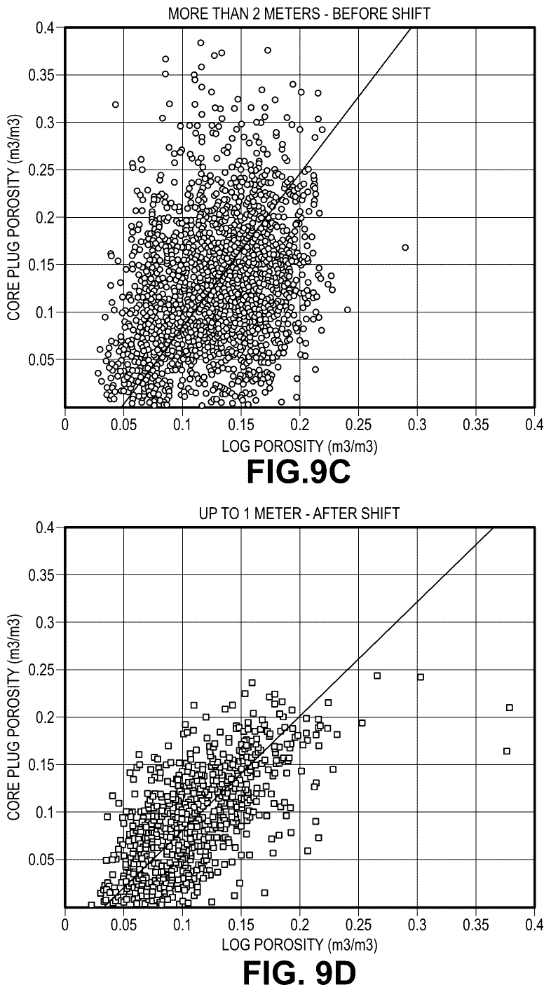

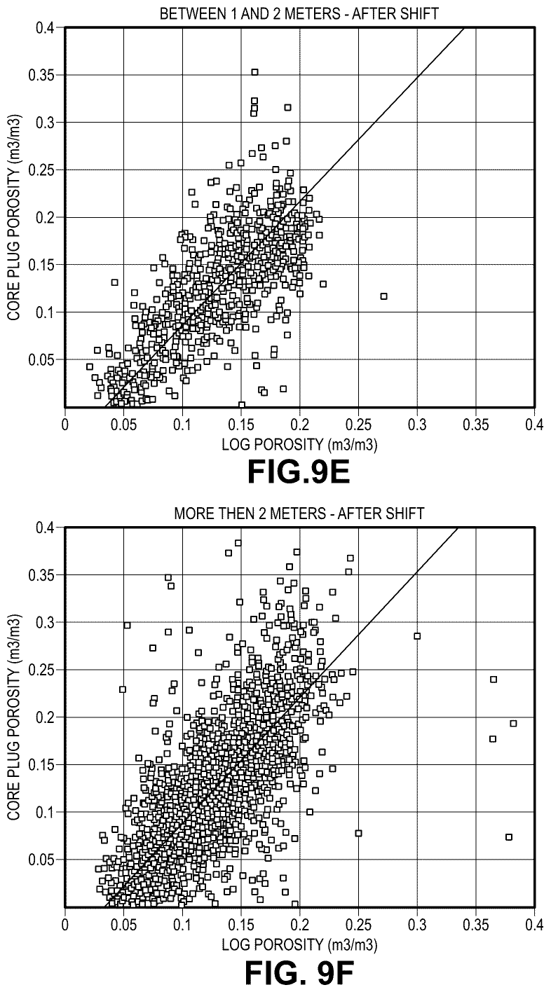

FIGS. 9A-9F illustrate cross-plots of core plug porosity (m³/m³) versus log porosity (m³/m³), according to an embodiment. More particularly, FIG. 9A shows samples shifted by up to 1 m before automated core-to-log depth shift, and FIG. 9D shows samples shifted by up to 1 m after automated core-to-log depth shift. FIG. 9B shows samples shifted between 1 and 2 m before automated core-to-log depth shift, and FIG. 9E shows samples shifted between 1 m and 2 m after automated core-to-log depth shift. FIG. 9C shows samples shifted by more than 1 m before automated core-to-log depth shift, and FIG. 9F shows samples shifted by more than 1 m after automated core-to-log depth shift.

The same effects of scale divergence observed for SWC are also seen in FIGS. 9A-9F. When the cross-plots before (FIGS. 9A, 9B, and 9C) and after (FIGS. 9D, 9E, and 9F) applying the proposed automated core-to-log depth matching algorithm are compared, one can notice that there is less spread in the points than at the original depth, notably for the core samples shifted by more than 2 m. The regression line of FIG. 9C tends to overestimate core porosity, presenting a divergent behavior of the overall data tendency.

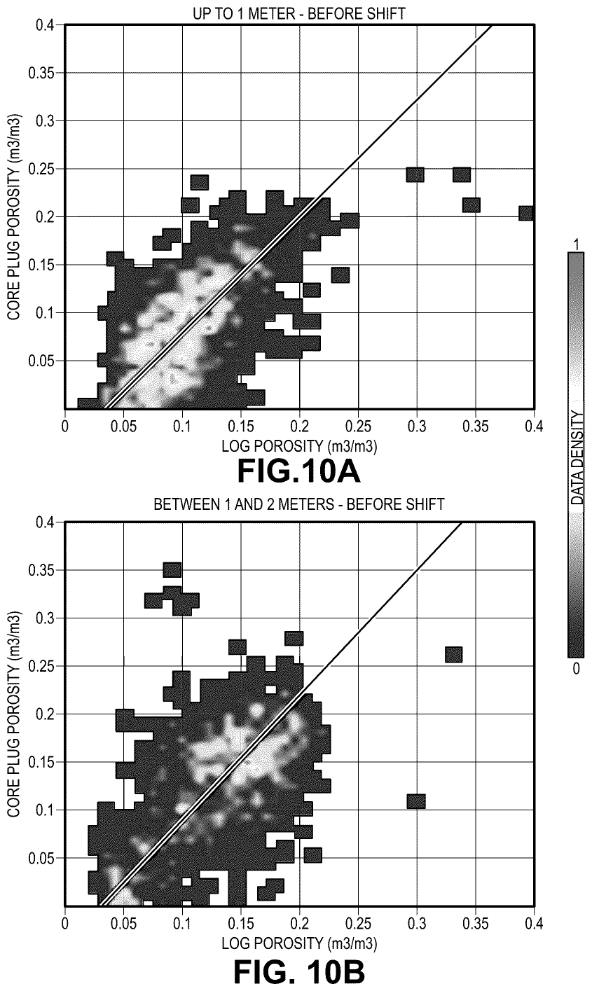

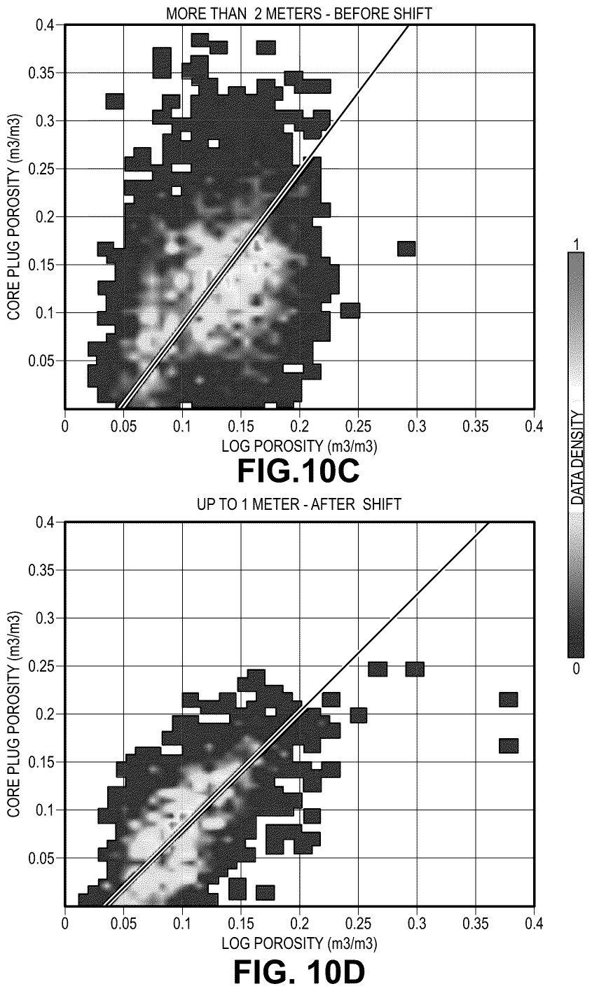

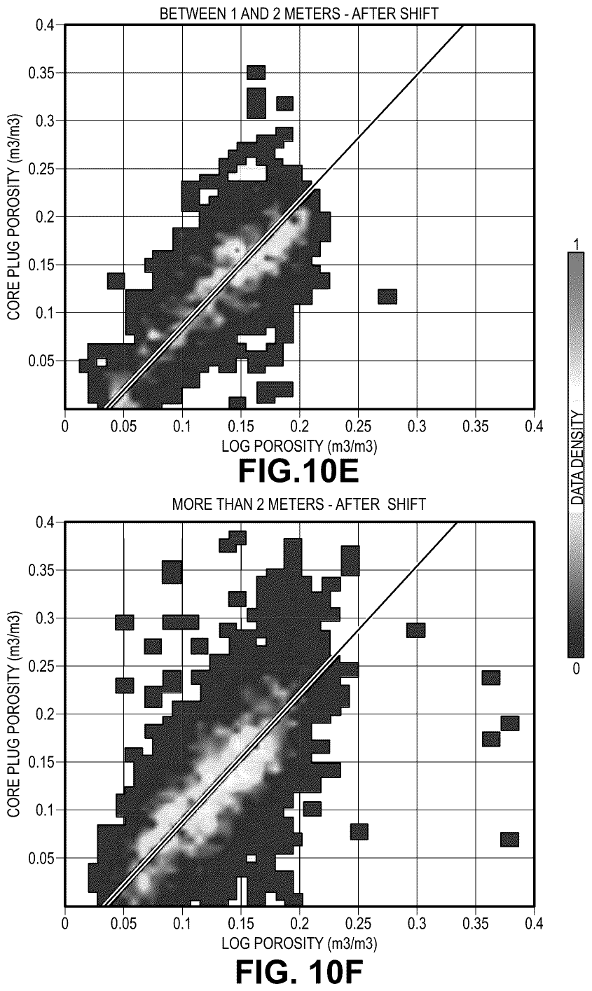

FIGS. 10A-10F illustrate density cross-plots of core plug porosity (m³/m³) versus log porosity (m³/m³), according to an embodiment. More particularly, FIG. 10A shows samples shifted by up to 1 m (a) before an automated core-to-log depth shift, and FIG. 10D shows samples shifted by up to 1 m after the automated core-to-log depth shift. FIG. 10B shows samples shifted between 1 and 2 m before an automated core-to-log depth shift, and FIG. 10E shows samples shifted between 1 and 2 m after the automated core-to-log depth shift. FIG. 10C shows samples shifted by more than 1 m before an automated core-to-log depth shift, and FIG. 10F shows samples shifted by more than 1 m after the automated core-to-log depth shift.

The data density of the cross-plots shows, for a qualitative evaluation, a high spread of data on the cross-plots of core plug porosity versus log porosity before depth correction. For those samples that used a large depth correction (e.g., more than 2-m-shift category), the improvement in the data density cross-plot is large. Before depth alignment, the density was spread along the regression (FIG. 10C), when compared to the higher densities along the regression after applying the proposed method (FIG. 10F).

### CONCLUSIONS

The proposed solution was validated at 37 wells located in 10 challenging Brazilian pre-salt fields. A total of 2135 SWC samples and 4836 core plug samples of pre-salt carbonates were submitted to an automated depth quality control. An average shift of 76 cm was used to correct the depth shift of SWC samples, while for core plug samples the average shift was 272 cm, indicating a lower precision on depth measurements of whole core sampling jobs. Due to this large shift on core plug samples, a greater improvement in the correlation of core and log data was observed for core plugs, where it was observed that the greatest the depth shift, the highest the improvement in core-to-log data correlation.

For this case study, core porosity values belonging to the same well can vary by up to 15% at the same depth. This level of formation heterogeneity on pre-salt carbonates highlights the value of a robust and automated method to adjust the core depths. Despite the relatively low correlation values between core and log porosity, ranging from approximately 0.33 to 0.59, it can be considered a good match between both measurements, given the data particularities related to acquisition, scales, and analyzed volumes. Compared to manual matching, the adoption of the data preprocessing strategy and an outlier removal procedure proved valuable in defining the actual depth of the samples with low to no user intervention.

Another valuable aspect of the proposed technique is the capability of saving time and effort, taking no more than 1 minute per well to automatically position the samples at their proper depth. Furthermore, the method may be applied to other scenarios, such as gamma ray measurements, with the advantage of being an automated approach. In addition to that, the core gamma ray measurements, acquired to perform depth control, may be omitted for pre-salt carbonate scenarios, reducing the cost of laboratory measurements.

The developed solution has proven to enhance the value of core samples for petrophysical models and improve overall accuracy, especially for carbonate reservoirs. The examples herein used core porosity data, but different laboratory measurements and sample descriptions may be carried out to the new suggested depth, such as permeability, X-ray diffractometry, and mercury injection capillary pressure. With this, the permeability, mineralogy, and water saturation models can achieve a greater accuracy and be extended to non-cored intervals.

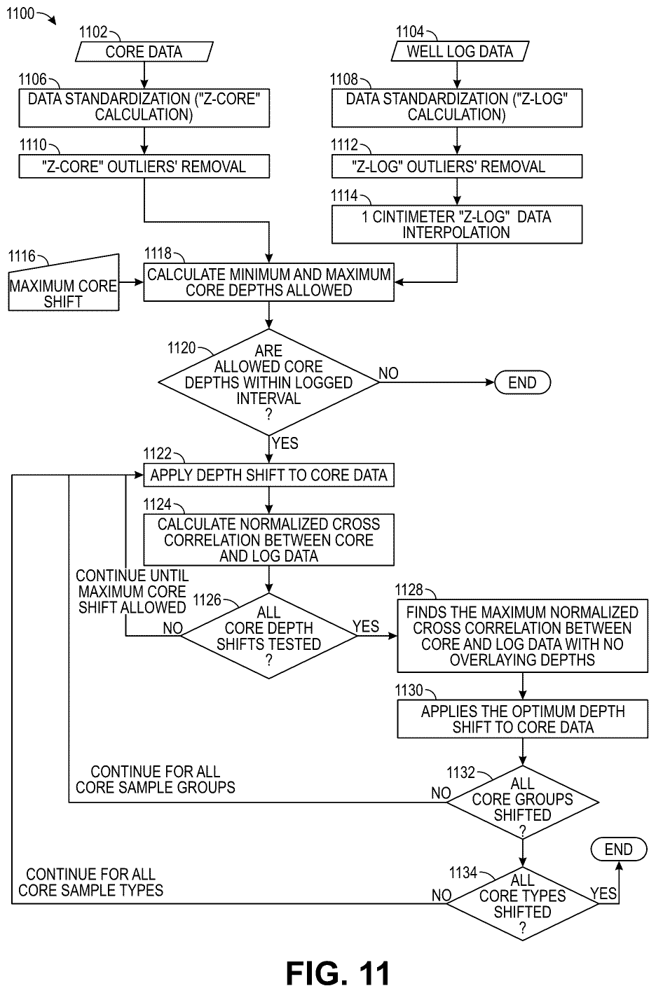

FIG. 11 illustrates a flowchart of a method 1100 for performing core-to-log depth matching, according to an embodiment. An illustrative order of the method 1100 is shown below; however, one or more portions of the method 1100 may be performed in a different order, simultaneously, repeated, or omitted.

The method 1100 may include receiving input data. The input data may include core data 1102 and well log data 1104. The core data may be measured from samples acquired by a first downhole tool within a wellbore. The well log data may be measured by sensors on a second downhole tool within the wellbore.

The method 1100 may also include performing data standardization of the core data 1106 and the well log data 1108. This may include performing an autonomous data preprocessing procedure to standardize the core data and the well log data to determine correlations between the core data and the well log data. The data standardization may be or include a z-core calculation.

The method 1100 may also include performing an autonomous outlier removal procedure to address differences in acquisition methods and measurement principles of the core data 1110 and the well log data 1112. This may include a z-core outlier removal for the core data, and a z-log outlier removal for the well log data.

The method 1100 may also include performing a (e.g., 1 cm) z-log data interpolation to the well log data, as at 1114.

The method 1100 may also include receiving or determining a maximum core shift, as at 1116.

The method 1100 may also include determining minimum and maximum core depths allowed, as at 1118. The core depths may be based upon the core data, the well log data, and/or the maximum core shift.

The method 1100 may also include determining that the allowed core depths are within a logged interval, as at 1120.

The method 1100 may also include applying a depth shift to the core data, as at 1122.

The method 1100 may also include automatically determining normalized cross-correlations between measurements derived from the core data and measurements derived from the well log data, as at 1124. The normalized cross-correlations may be based upon the correlations between the core data and the well log data. The normalized cross-correlations may be determined within a predetermined depth shift interval in the wellbore.

The method 1100 may also include determining that the core depth shifts are tested, as at 1126.

The method 1100 may also include identifying a maximum normalized cross-correlation between the core data and the well log data with no overlying depths, as at 1128.

The method 1100 may also include applying the optimum depth shift to the core data, as at 1130. This may include automatically shifting the measurements derived from the core data to a new depth position based upon a maximum of the normalized cross-correlations, to complete the core-to-log depth matching with no user intervention.

The method 1100 may also include determining that each of the core groups are shifted, as at 1132.

The method 1100 may also include determining that each of the core types are shifted, as at 1134.

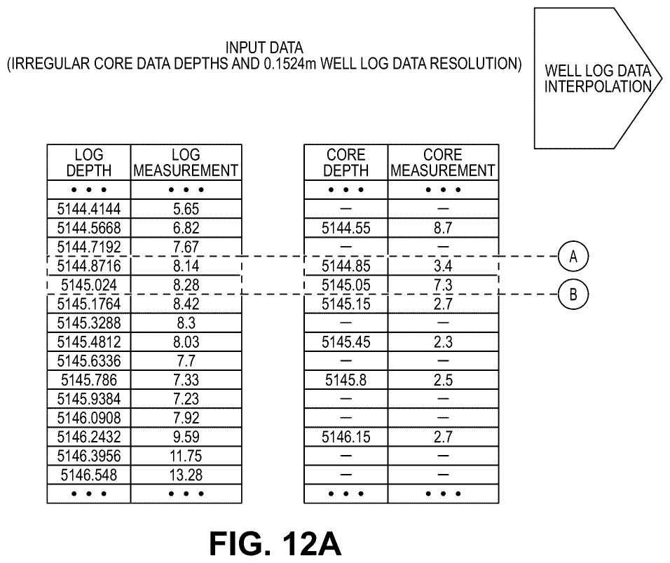

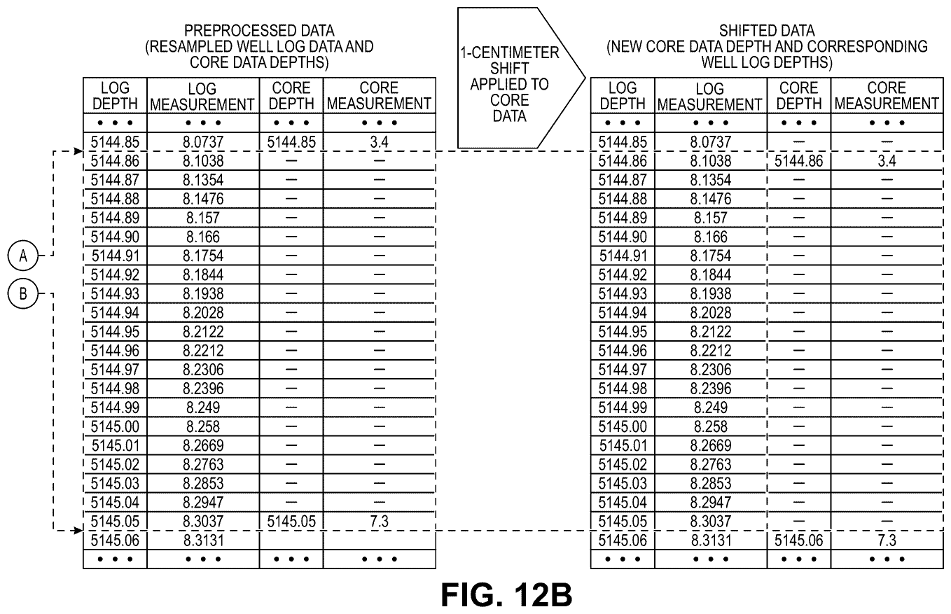

FIGS. 12A and 12B illustrate well log data resampling to allow that, at each 1 cm shift applied to the core data, it can be correlated with measurements derived from the well log data at the same depth, according to an embodiment.

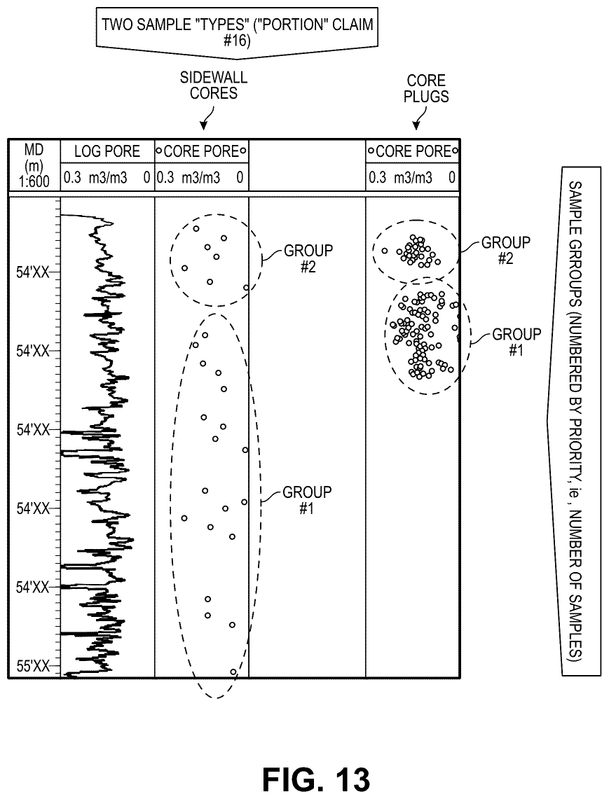

FIG. 13 illustrates two different groups of two types of samples (e.g., sidewall cores and core plugs), according to an embodiment.

It should be apparent from the foregoing description that various exemplary embodiments of the disclosure may be implemented in hardware. Furthermore, various exemplary embodiments may be implemented as instructions stored on a non-transitory machine-readable storage medium, such as a volatile or non-volatile memory, which may be read and executed by at least one processor to perform the operations described in detail herein. A machine-readable storage medium may include any mechanism for storing information in a form readable by a machine, such as a personal or laptop computer, a server, or other computing device. Thus, a non-transitory machine-readable storage medium excludes transitory signals but may include both volatile and non-volatile memories, including but not limited to read-only memory (ROM), random-access memory (RAM), magnetic disk storage media, optical storage media, flash-memory devices, and similar storage media.

It should be appreciated by those skilled in the art that any figures, diagrams, and charts described herein represent conceptual views of illustrative embodiments of the principles of the present disclosure. Similarly, it will be appreciated that any flow charts, flow diagrams, state transition diagrams, pseudo code, and the like represent various processes which may be substantially represented in machine readable media and so executed by a computer or processor, whether or not such computer or processor is explicitly shown.

It will be appreciated that various implementations of the above-disclosed and other features and functions, or alternatives or varieties thereof, may be desirably combined into many other different systems or applications. Also, that various presently unforeseen or unanticipated alternatives, modifications, variations or improvements therein may be subsequently made by those skilled in the art which are also intended to be encompassed by the following claims.

The foregoing description, for purposes of explanation, has been described with reference to specific embodiments. However, the illustrative discussions above are not intended to be exhaustive or limiting to the precise forms disclosed. Many modifications and variations are possible in view of the above teachings. Moreover, the order in which the elements of the methods described herein are illustrated and described may be re-arranged, and/or two or more elements may occur simultaneously. The embodiments were chosen and described in order to best explain the principles of the disclosure and its practical applications, to thereby enable others skilled in the art to best utilize the disclosed embodiments and various embodiments with various modifications as are suited to the particular use contemplated.

What is claimed is:

1. A method for performing core-to-log depth matching, the method comprising:
receiving input data, wherein the input data comprises core data and well log data, wherein the core data is measured from samples acquired by a first downhole tool within a wellbore, and wherein the well log data is measured by sensors on a second downhole tool within the wellbore;
performing an autonomous data preprocessing procedure to standardize the core data and the well log data to determine correlations between the core data and the well log data;
performing an autonomous outlier removal procedure to address differences in acquisition methods and measurement principles of the core data and the well log data;
automatically determining normalized cross-correlations between measurements derived from the core data and measurements derived from the well log data based upon the correlations between the core data and the well log data, wherein the normalized cross-correlations are determined within a predetermined depth shift interval in the wellbore; and
automatically shifting the measurements derived from the core data to a new depth position based upon a maximum of the normalized cross-correlations, to complete the core-to-log depth matching with no user intervention.

2. The method of claim 1, wherein the measurements derived from the core data comprise a plurality of first measurements, wherein the measurements derived from the well log data comprises a plurality of second measurements, and wherein the first measurements are different than the second measurements.

3. The method of claim 1, wherein performing the autonomous data preprocessing procedure comprises standardizing the core data and the well log data, separately, by automatically applying a z-score metric using:
$$Z = \frac{( x - μ )}{σ}$$
where Z represents the z-score metric, x represents values of the measurements derived from the core data and the measurements derived from the well log data, μ represents average values of the core data and average values of the well log data, and σ represents a standard deviation of the core data and of the well log data.

4. The method of claim 3, wherein performing the autonomous data preprocessing procedure comprises applying the z-score metric on the core data and the well log data to:
account for different sampling rates and/or volumes of interest in the core data and the well log data;
compare distinct yet interrelated physical properties in the core data and the well log data; and
accentuate variations in magnitude for properties with a narrow range of values in the core data and the well log data, making small differences more noticeable and comparable.

5. The method of claim 1, wherein performing the autonomous data preprocessing procedure comprises resampling the well log data to a 1-centimeter resolution using linear interpolation to increase the resolution of the well log data, and wherein performing the autonomous data preprocessing procedure comprises:
resampling the well log data to allow that, for each 1-centimeter shift applied to core data, a depth is mapped onto the well log data; and
determining the correlation based upon the mapped depth.

6. The method of claim 1, wherein performing the autonomous outlier removal procedure comprises removing outlying values in the well log data and the core data to address:
differences in data resolution between the core data and the well log data;
adverse conditions during construction of the wellbore and extraction of the samples; and
errors in the measurements derived from the core data related to core cleaning and core expansion at the surface, due to pressure release.

7. The method of claim 1, wherein the autonomous outlier removal procedure adopts two units of standard deviation as z-score cutoff values for the core data and the well log data.

8. The method of claim 1, wherein determining the normalized cross-correlations is limited to a maximum shift to the core data, and wherein determining the normalized cross-correlations automatically verifies whether the maximum shift to the core data will result in the new depth position being within the predetermined depth shift interval.

9. The method of claim 1, wherein the normalized cross-correlations are utilized as metrics to measure similarities between two signals associated with the core data and the well log data, respectively, using:
$$NCC = \frac{{\sum }_{d} \{ [ A ( d ) - \overline{A} ] * [ B ( d - s ) - \overline{B} ] \}}{\sqrt{{\sum }_{d} \{ {[ A ( d ) - \overline{A} ]}^{2} * {[ B ( d - s ) - \overline{B} ]}^{2} \}}} \qquad ( 2 )$$
where NCC represents the metrics of the normalized cross-correlations, A represents z-score values of the well log data, A represents averages of the z-score values of the well log data, B represents z-score values of the core data, B represents averages of the z-score values of the core data, d represents a depth shift applied to core data to reach the new depth position, ranging from minimum to maximum within the predetermined depth shift interval, and s represents the maximum shift to the core data.

10. The method of claim 9, wherein the normalized cross-correlations use a metric to account for differences in a mean and a standard deviation of the measurements derived from the core data and the measurements derived from the well log data, which use distinct measurement principles.

11. The method of claim 9, wherein the normalized cross-correlations provide a quantitative measurement of similarity between the core data and the well log data, enabling an objective assessment of the core-to-log depth matching.

12. The method of claim 1, wherein the normalized cross-correlations are determined for a plurality of possible depths within the predetermined depth shift interval, and wherein determining the normalized cross-correlations comprises automatically determining an optimum depth position according to the maximum of the normalized cross-correlations and shifting the measurements derived from the core data to the optimum depth position.

13. The method of claim 12, wherein automatically determining the normalized cross-correlations comprises separately determining the optimum depth position and shifting distinct groups of the samples from the wellbore to:
differentiate a first type of the samples from a second type of the samples, wherein the first type comprises sidewall core samples, and wherein the second type comprises core plug samples; and
minimize depth errors that arise from core fragmentation in unconsolidated, highly porous or fractured formations during acquisition of the core plug samples.

14. The method of claim 13, wherein separately determining the optimum depth position for each type of the samples prioritizes the groups with greater statistical representativeness, starting by shifting the groups with larger numbers of the samples before shifting groups with smaller numbers of the samples.

15. The method of claim 14, wherein independently shifting the groups with the larger numbers of the samples before shifting the groups with the smaller numbers of the samples does not allow two groups of the first type of the samples to share the same depths, independent of the normalized cross-correlations.

16. The method of claim 14, wherein independently shifting the groups with the larger numbers of the samples before shifting the groups with the smaller numbers of the samples automatically identifies a second best maximum of the normalized cross-correlations at a depth which does not overlay another of the groups with a greater priority, for the first type of samples.

17. The method of claim 14, wherein independently shifting the groups with the larger numbers of the samples before shifting the groups with the smaller numbers of the samples allows two groups of the second type of the samples to share the same depths, independent of the normalized cross-correlations.

18. The method of claim 14, wherein separately determining the optimum depth position for each of the groups of samples differentiates the first type of the samples from the second type of the samples, enabling the two types of samples to share the same depths.

19. A computing system, comprising:
one or more processors; and
a memory system comprising one or more non-transitory computer-readable media storing instructions that, when executed by at least one of the one or more processors, cause the computing system to perform operations, the operations comprising:
receiving input data, wherein the input data comprises core data and well log data, wherein the core data is measured from samples acquired by a first downhole tool within a wellbore, wherein the samples comprise core plug samples and/or sidewall core samples, and wherein the well log data is measured by sensors on a second downhole tool within the wellbore;
performing an autonomous data preprocessing procedure to standardize the core data and the well log data to determine correlations between the core data and the well log data;
performing an autonomous outlier removal procedure to address differences in acquisition methods and measurement principles of the core data and the well log data;
automatically determining normalized cross-correlations between measurements derived from the core data and measurements derived from the well log data based upon the correlations between the core data and the well log data, wherein the normalized cross-correlations are determined within a predetermined depth shift interval in the wellbore; and
automatically shifting the measurements derived from the core data to a new depth position based upon a maximum of the normalized cross-correlations, to complete the core-to-log depth matching with no user intervention.

20. A non-transitory computer-readable medium storing instructions that, when executed by one or more processors of a computing system, cause the computing system to perform operations, the operations comprising:
receiving input data, wherein the input data comprises core data and well log data, wherein the core data is measured from samples acquired by a first downhole tool within a wellbore, and wherein the well log data is measured by sensors on a second downhole tool within the wellbore;
performing an autonomous data preprocessing procedure to standardize the core data and the well log data to determine correlations between the core data and the well log data;
performing an autonomous outlier removal procedure to address differences in acquisition methods and measurement principles of the core data and the well log data;
automatically determining normalized cross-correlations between measurements derived from the core data and measurements derived from the well log data based upon the correlations between the core data and the well log data, wherein depths corresponding to the measurements derived from the well log data serve as static depth references, wherein depths corresponding to the measurements derived from the core data are uncertain, wherein the normalized cross-correlations are determined within a predetermined depth shift interval in the wellbore, and wherein determining the normalized cross-correlations interactively shifts and determines correlation metrics for a plurality of possible depths within the predetermined depth shift interval; and
automatically shifting the measurements derived from the core data to a new depth position based upon a maximum of the normalized cross-correlations, to complete the core-to-log depth matching with no user intervention.
